(19) United States
(12) Patent Application Publication
(10) Pub. No.: US 2015/0265461 A1
(43) Pub. Date: Sep. 24, 2015

(54) COUGH SUPPRESSANT GARMENT AND SYSTEM AND METHOD FOR SUPPRESSING COUGHING

(71) Applicant: Angel W. Marquez, Chicago, IL (US)

(72) Inventor: Angel W. Marquez, Chicago, IL (US)

(21) Appl. No.: 14/716,306

(22) Filed: May 19, 2015

Related U.S. Application Data
(62) Division of application No. 13/367,923, filed on Feb. 7, 2012.

(51) Int. Cl. A61F 7/08

(52) U.S. Cl. CPC A61F 7/086; A61F 2007/0011

(57) ABSTRACT

A cough suppressant garment may be worn around the neck of the user. The cough suppressant garment may have three layers, and one or more of the three layers may be manufactured from a woven material, such as, for example, cotton. An end of the cough suppressant garment may reversibly attach to an opposite end of the cough suppressant garment to maintain a position of the cough suppressant garment on the neck of the user.

### CROSS REFERENCE TO RELATED APPLICATION

This application is a Divisional Application of and claims the benefit of co-pending U.S. patent application Ser. No. 13/367,923 filed on Feb. 7, 2012.

### BACKGROUND OF THE INVENTION

The present invention generally relates to a cough suppressant garment and a system and a method for suppressing coughing. More specifically, the present invention relates to a cough suppressant garment which may be worn around the neck of a user.

Coughing is a mechanism for clearing secretions, irritants, foreign particles and microbes from the airways of a person, such as the throat of the person. However, coughing is typically repetitive and may be uncomfortable, painful and stressful. For example, coughing may impair breathing and may prevent sleep. Further, coughing may disrupt the person's activities and may disturb nearby individuals, such as co-workers, friends and family. Moreover, persistent coughing is a common reason for visiting a physician, and visiting a physician may be inconvenient and may be expensive.

It is well known to use throat lozenges, cough syrups, cough drops, throat sprays or decongestants for relief of coughing. However, these treatments are often ineffective and may have adverse consequences for pregnant women, children or the elderly. Prescription drugs may be used for relief of coughing, and prescription drugs are typically more effective than throat lozenges, cough syrups, cough drops, throat sprays and decongestants. However, prescription drugs are expensive, may require the user to follow detailed instructions, may have undesired side effects and may interact with other medications. A need, therefore, exists for a system and a method for suppressing coughing which do not require the user to ingest prescription drugs, throat lozenges, cough syrups, cough drops throat sprays or decongestants.

### SUMMARY OF THE INVENTION

The present invention generally relates to a cough suppressant garment and a system and a method for suppressing coughing. More specifically, the present invention relates to a cough suppressant garment which may be worn around the neck of the user. The cough suppressant garment may have three layers, and one or more of the three layers may be manufactured from a woven material, such as, for example, cotton.

To this end, in an embodiment of the present invention, a garment worn on a neck of a user to suppress coughing of the user is provided. The garment has a first layer of woven material wherein the first layer has a first edge, a second edge, a third edge and a fourth edge and further wherein the first edge is located in a position opposite to the second edge, the third edge is located in a position opposite to the fourth edge, the first edge is parallel to the second edge, and the third edge is parallel to the fourth edge wherein the first edge and the second edge are perpendicular to the third edge and the fourth edge; a second layer of woven material wherein the second layer has a first edge, a second edge, a third edge and a fourth edge and further wherein the first edge is located in a position opposite to the second edge, the third edge is located in a position opposite to the fourth edge, the first edge is parallel to the second edge, and the third edge is parallel to the fourth edge wherein the first edge and the second edge are perpendicular to the third edge and the fourth edge and further wherein the first edge of the second layer is integral with the first edge of the first layer; an insert located between the first layer and the second layer wherein the insert extends from the first edge of the first layer to the second edge of the first layer and further wherein the insert extends from the third edge of the first layer to the fourth edge of the first layer; thread which connects the second edge of the first layer to the second edge of the second layer, connects the third edge of the first layer to the third edge of the second layer, and connects the fourth edge of the first layer to the fourth edge of the second layer; first male fasteners which extend in a direction generally outward from the first layer wherein the first male fasteners are located in a position adjacent to the first edge of the first layer and the first edge of the second layer; and first female fasteners which extend in a direction generally inward into the second layer wherein the first female fasteners are located in a position adjacent to the second edge of the first layer and the second edge of the second layer and further wherein the first male fasteners insert into the first female fasteners to connect the first edge of the first layer and the first edge of the second layer to the fourth edge of the first layer and the fourth edge of the second layer wherein the first male fasteners separate from the first female fasteners to separate the first edge of the first layer and the first edge of the second layer from the fourth edge of the first layer and the fourth edge of the second layer.

In an embodiment, the insert is made from woven material.

In an embodiment, the first layer, the insert and the second layer are approximately 100% cotton.

In an embodiment, the insert has a substantially rectangular shape.

In an embodiment, the garment has second male fasteners located at a greater distance from the first edge of the first layer and the first edge of the second layer relative to the first male fasteners wherein the second male fasteners extend in a direction generally outward from the first layer.

In an embodiment, the garment has second female fasteners located at a greater distance from the second edge of the first layer and the second edge of the second layer relative to the first female fasteners wherein the second female fasteners extend in a direction generally inward into the second layer.

In an embodiment, the first edge of the first layer, the second edge of the first layer, the first edge of the second layer, and the second edge of the second layer have a length between approximately 2.5 inches and approximately 3.0 inches.

In an embodiment, the third edge of the first layer, the fourth edge of the first layer, the third edge of the second layer, and the fourth edge of the second layer have a length between approximately fifteen inches and approximately twenty-four inches.

In an embodiment, the first layer, the insert and the second layer form three layers and further wherein the three layers are the only layers of the garment.

In an embodiment, the garment has a lip located in each of the female fasteners wherein each of the male fasteners has a groove wherein the lip inserts into the groove to connect the female fasteners to the male fasteners and further wherein the lip separates from the groove to separate the female fasteners from the male fasteners.

In another embodiment of the present invention, a system for suppressing coughing of a user having a neck is provided. The system has a garment having a first edge, a second edge, a third edge and a fourth edge wherein the first edge is located in a position opposite to the second edge, the third edge is located in a position opposite to the fourth edge, the first edge is parallel to the second edge, and the third edge is parallel to the fourth edge and further wherein the first edge and the second edge are perpendicular to the third edge and the fourth edge wherein the garment has a first side and a second side and further wherein the first side is located in a position opposite to the second side wherein the first side contacts the neck of the user; an insert located within the garment wherein the insert is located between the first side and the second side of the garment and further wherein the insert extends from the first edge to the second edge and further wherein the insert extends from the third edge to the fourth edge; a first hook-and-loop portion attached to the first side of the garment wherein the first hook-and-loop portion is located adjacent to the first edge; and a second hook-and-loop portion attached to the second side of the garment wherein the second hook-and-loop portion is located adjacent to the second edge wherein the first hook-and-loop portion connects to the second hook-and-loop portion to connect the first edge to the second edge and further wherein the first hook-and-loop portion separates from the second hook-and-loop portion to separate the first edge from the second edge.

In an embodiment, the garment has a rectangular shape wherein the first edge, the second edge, the third edge and the fourth edge are the only edges of the garment.

In an embodiment, the garment is made of woven material.

In an embodiment, the system has thread connected to the garment wherein the garment is formed from one piece of material having a midline, a first end and a second end and further wherein the midline forms the first edge of the garment wherein the thread connects the first end of the one piece of material to the second end of the one piece of material to form the second edge of the garment.

In another embodiment of the present invention, a method for suppressing coughing of a user having a neck is provided. The method has the steps of placing a garment having a rectangular shape on the neck of the user wherein the garment has a first end and a second end and further wherein the first end is located in a position opposite to the second end wherein first connecting means are attached to the first end and second connecting means are attached to the second end and further wherein the first connecting means and the second connecting means are attached to opposite sides of the garment; connecting the first connecting means to the second connecting means wherein connecting the first connecting means to the second connecting means attaches the first end to the second end; separating the first connecting means from the second connecting means wherein separating the first connecting means from the second connecting means separates the first end from the second end; and removing the garment from the neck of the user.

In an embodiment, the garment has three layers of woven material and further wherein the three layers of woven material are the only layers of the garment.

In an embodiment, the method has the step of connecting the first connecting means to third connecting means wherein the third connecting means are attached to the second end of the garment and further wherein a radius of the garment is smaller when the first connecting means are connected to the third connecting means instead of the second connecting means.

In an embodiment, the method has the step of inserting male fasteners into female fasteners to attach the first end to the second end wherein separating the male fasteners from the female fasteners separates the first end from the second end and further wherein the first connecting means are the male fasteners and the second connecting means are the female fasteners.

In an embodiment, the method has the step of connecting a first hook-and-loop portion to a second hook-and-loop portion to attach the first end to the second end wherein separating the first hook-and-loop portion from the second hook-and-loop portion separates the first end from the second end and further wherein the first connecting means is the first hook-and-loop portion and the second connecting means is the second hook-and-loop portion.

In an embodiment, the method has the step of connecting a magnet to a portion containing iron to attach the first end to the second end wherein separating the magnet from the portion containing iron separates the first end from the second end and further wherein the first connecting means is the magnet and the second connecting means is the portion containing iron.

It is, therefore, an advantage of the present invention to provide a cough suppressant garment and a system and a method for suppressing coughing.

Another advantage of the present invention is to provide a cough suppressant garment and a system and a method for suppressing coughing which do not require the user to ingest prescription drugs, throat lozenges, cough syrups, cough drops throat sprays or decongestants.

Yet another advantage of the present invention is to provide a cough suppressant garment and a system and a method for suppressing coughing which may be easily and quickly worn when needed and may be easily and quickly removed when no longer needed.

A still further advantage of the present invention is to provide a cough suppressant garment and a system and a method for suppressing coughing which may suppress coughing for pregnant women, children and the elderly.

Another advantage of the present invention is to provide a cough suppressant garment and a system and a method for suppressing coughing which may be easily cleaned.

Yet another advantage of the present invention is to provide a cough suppressant garment and a system and a method for suppressing coughing which have connections that maintain a position on the garment on the neck of a user.

A still further advantage of the present invention is to provide a cough suppressant garment and a system and a method for suppressing coughing which may have one or more layers of woven material.

Another advantage of the present invention is to provide a cough suppressant garment and a system and a method for suppressing coughing which may be worn without interfering with activities of the user.

And another advantage of the present invention is to provide a cough suppressant garment and a system and a method for suppressing coughing which may have connections that enable the diameter of the garment to be changed.

Still further, an advantage of the present invention is to provide a cough suppressant garment and a system and a method for suppressing coughing which may not have side effects or interact with medication.

Another advantage of the present invention is to provide a cough suppressant garment and a system and a method for suppressing coughing which may be reusable.

Moreover, an advantage of the present invention is to provide a cough suppressant garment and a system and a method for suppressing coughing which may be manufactured from only two pieces of material.

Additional features and advantages of the present invention are described in, and will be apparent from, the detailed description of the presently preferred embodiments and from the drawings.

### BRIEF DESCRIPTION OF THE DRAWINGS

FIG. 1 illustrates a side plan view of a cough suppressant garment in an embodiment of the present invention.

FIG. 2 illustrates a side plan view of a cough suppressant garment in an embodiment of the present invention from an opposite side relative to FIG. 1.

FIG. 3 illustrates a top plan view of a cough suppressant garment in an embodiment of the present invention.

FIG. 4 illustrates a bottom plan view of a cough suppressant garment in an embodiment of the present invention.

FIGS. 5 and 6 illustrate perspective views of a cough suppressant garment in an embodiment of the present invention.

FIG. 7 illustrates a perspective view of fasteners of a cough suppressant garment in an embodiment of the present invention.

FIG. 8 illustrates a perspective view of a cough suppressant garment in an embodiment of the present invention.

FIG. 9 illustrates a side plan view of a cough suppressant garment in an embodiment of the present invention.

FIG. 10 illustrates a side plan view of a cough suppressant garment in an embodiment of the present invention from an opposite side relative to FIG. 9.

FIG. 11 illustrates a top plan view of a cough suppressant garment in an embodiment of the present invention.

FIG. 12 illustrates a bottom plan view of a cough suppressant garment in an embodiment of the present invention.

FIGS. 13 and 14 illustrate perspective views of a cough suppressant garment in an embodiment of the present invention.

FIG. 15 illustrates a side plan view of a cough suppressant garment in an embodiment of the present invention.

FIG. 16 illustrates a side plan view of a cough suppressant garment in an embodiment of the present invention from an opposite side relative to FIG. 15.

FIG. 17 illustrates a side plan view of a cough suppressant garment in an embodiment of the present invention.

FIG. 18 illustrates a perspective view of a cough suppressant garment in an embodiment of the present invention.

### DETAILED DESCRIPTION OF THE PRESENTLY PREFERRED EMBODIMENTS

The present invention generally relates to a cough suppressant garment and a system and a method for suppressing coughing. More specifically, the present invention relates to a cough suppressant garment which may be worn around the neck of the user. The cough suppressant garment may have three layers, and one or more of the three layers may be manufactured from a woven material, such as, for example, cotton, and/or a man-made material. An end of the cough suppressant garment may reversibly attach to an opposite end of the cough suppressant garment to maintain a position of the cough suppressant garment on the neck of the user. For example, hook-and-loop fasteners, snaps, buttons, magnets, hooks, a buckle, laces and/or the like may attach one end of the cough suppressant garment to the opposite end of the cough suppressant garment. The three layers of the garment may provide warmth to and/or may increase the temperature of the neck and the throat of the user. The warmth may thin the mucus in the throat, and thinning the mucus in the throat may suppress and/or may prevent coughing and/or may naturally lubricate the throat.

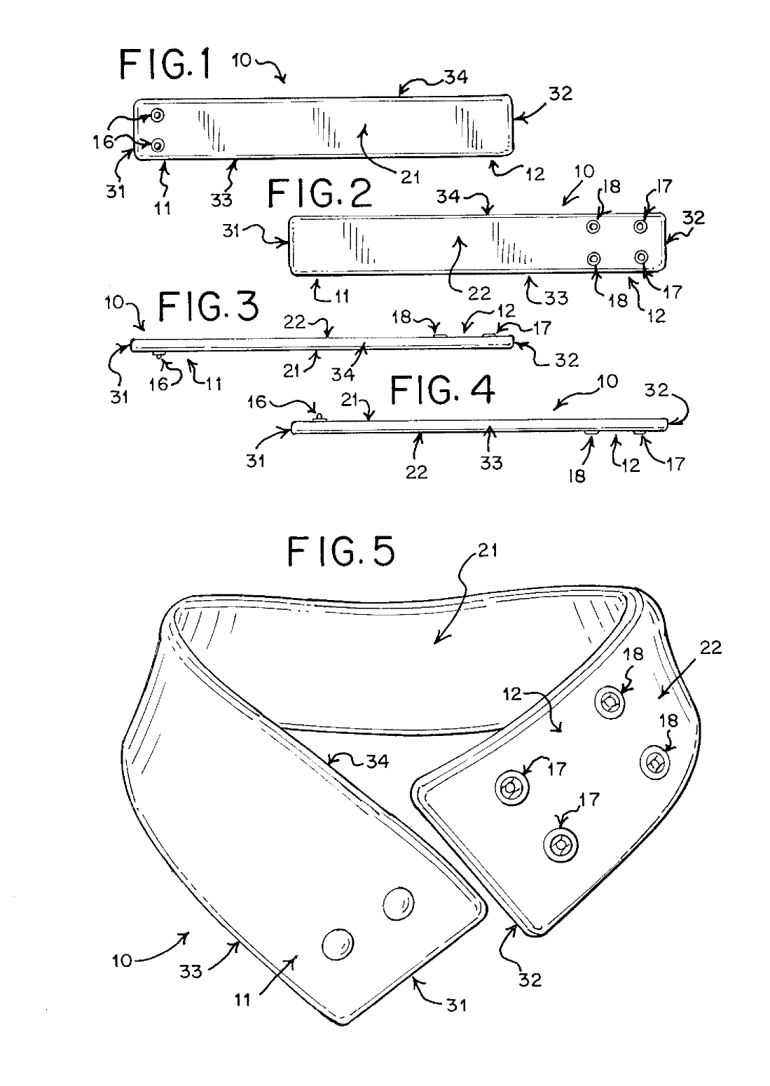

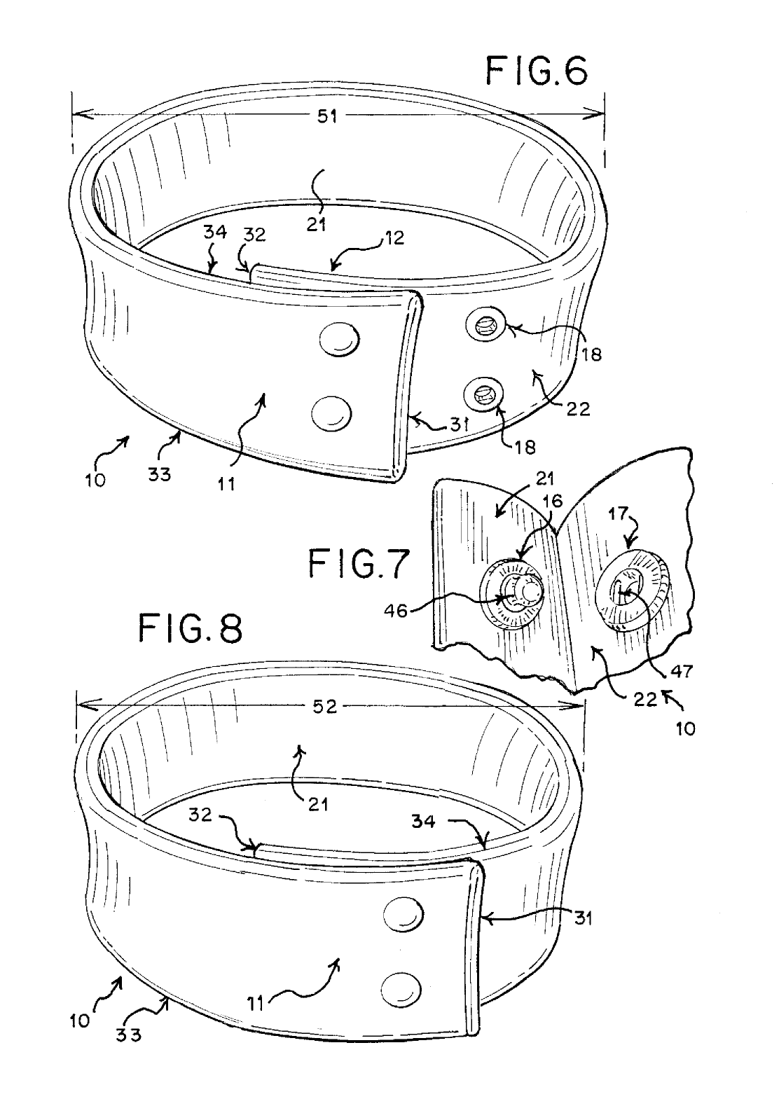

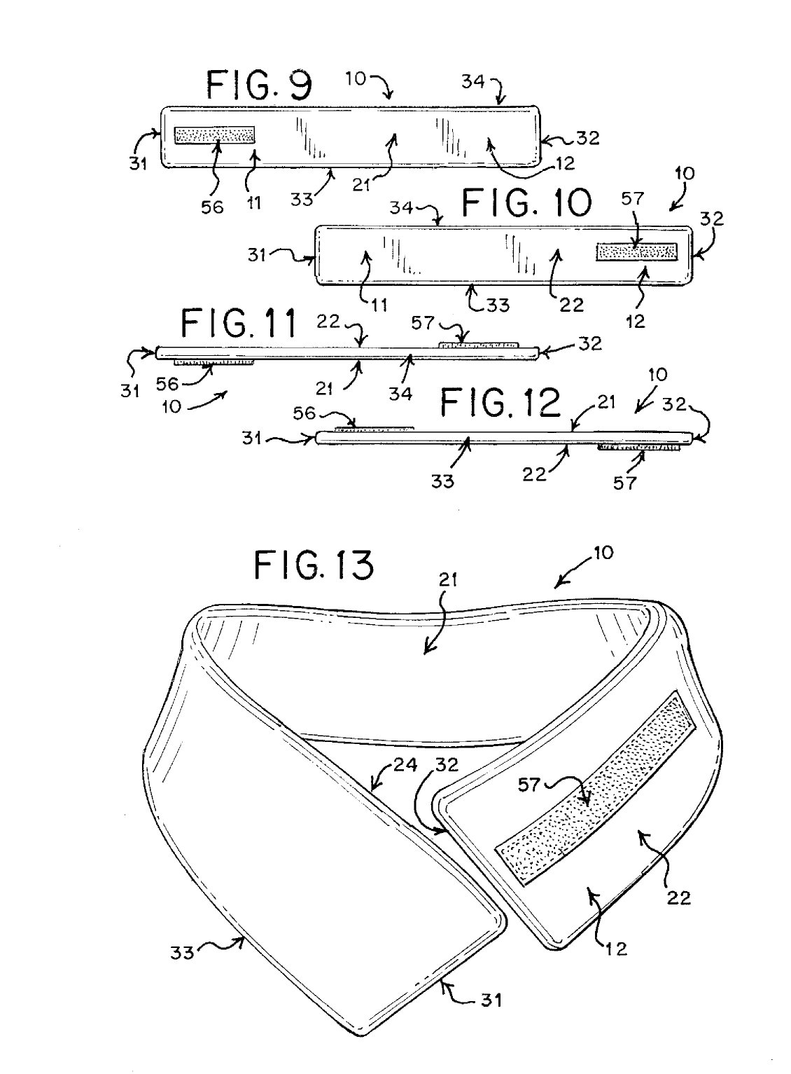

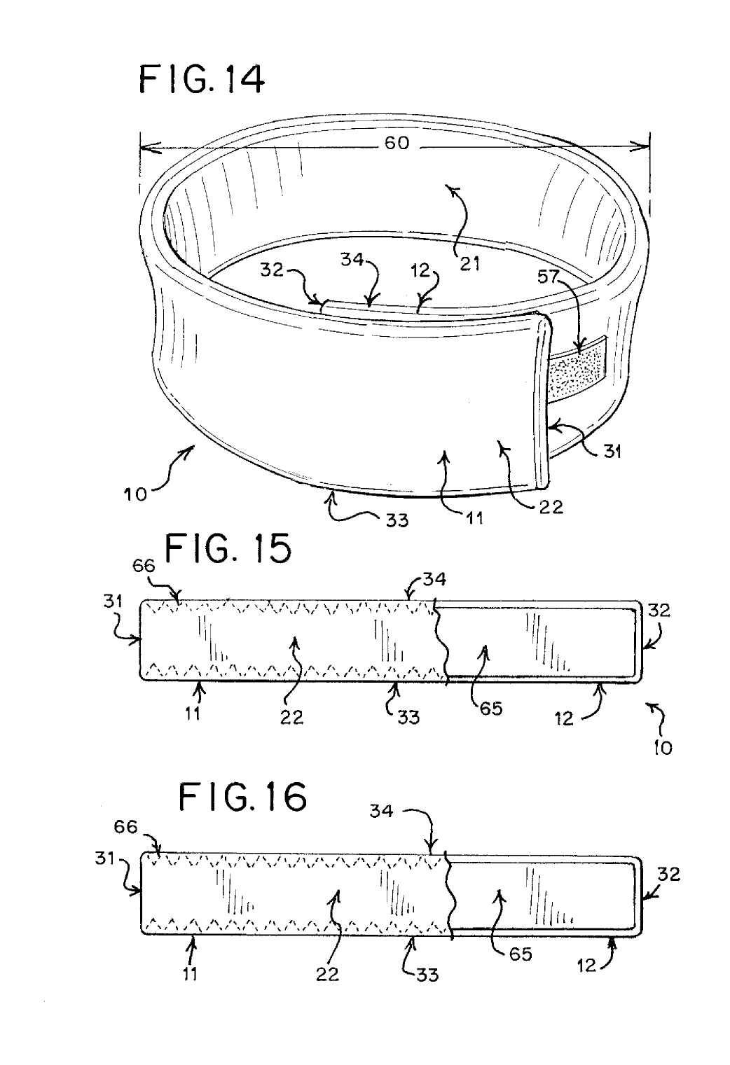

Referring now to the drawings wherein like numerals refer to like parts, FIGS. 1-18 generally illustrate a cough suppressant garment 10 (hereinafter “the garment 10”) in an embodiment of the present invention. The garment 10 may have a first end 11 and/or a second end 12, and the first end 11 may be located in a position opposite to the second end 12. The garment 10 may have a first edge 31 located at the first end 11 and/or may have a second edge 32 located at the second end 12. In an embodiment, the first edge 31 and the second edge 32 may be approximately the same length. In an embodiment, the first edge 31 and/or the second edge 32 may be between approximately two and a half inches long and approximately three inches long.

The garment 10 may have a third edge 33 which may extend from the first edge 31 to the second edge 32. The garment 10 may have a fourth edge 34 which may be located in a position opposite to the third edge 33 and/or may extend from the first edge 31 to the second edge 32. In an embodiment, the third edge 33 and the fourth edge 34 may be approximately the same length. In an embodiment, the third edge 33 and/or the fourth edge 34 may be between approximately fifteen inches long and approximately twenty-four inches long. The first edge 31, the second edge 32, the third edge 33 and the fourth edge 34 of the garment 10 may be any length, and the present invention is not limited to a specific length of the first edge 31, the second edge 32, the third edge 33 and the fourth edge 34 of the garment 10.

The garment 10 may have a first side 21 and/or a second side 22, and the first side 21 may be located in a position opposite to the second side 22. If the garment 10 is worn on the neck of a user, the first end 11 of the garment 10 may be located adjacent to the second end 12 of the garment 10, and/or the first side 21 of the garment 10 may contact the neck of the user.

In an embodiment, the first side 21 of the garment 10 and/or the second side 22 of the garment 10 may be rectangular. For example, the third edge 33 and the fourth edge 34 may be parallel to each other and/or may be perpendicular to the first edge 31 and/or the second edge 32. The first edge 31 and the second edge 32 of the garment 10 may be parallel to each other and/or may be perpendicular to the third edge 33 and/or the fourth edge 34 of the garment 10. In an embodiment, the first edge 31, the second edge 32, the third edge 33 and the fourth edge 34 may be the only edges of the garment 10. The first side 21 of the garment 10 and/or the second side 22 of the garment 10 may have any shape, and the present invention is not limited to a specific shape of the first side 21 of the garment 10 and the second side 22 of the garment 10.

As generally illustrated in FIGS. 6 and 8, the first end 11 of the garment 10 may reversibly connect to the second end of the garment 10 as discussed in more detail hereafter. For example, the user may determine that use of the garment 10 is desired, such as, for example, if the user is coughing. Then, the garment 10 may be placed around the neck of the user. The first end 11 of the garment 10 may then be connected to the second end 12 of the garment 10 to maintain a position of the garment 10 on the neck of the user. Subsequently, the user may determine that use of the garment 10 is no longer desired, such as, for example, if coughing ceases. Then, the first end 11 of the garment 10 may be separated from the second end 12 of the garment 10. The user may then remove the garment 10 from the neck of the user.

Referring again to FIGS. 1-6, the garment 10 may have one or more first fasteners 16 (hereinafter “the first fasteners 16”) and/or one or more second fasteners 17 (hereinafter “the second fasteners 17”). For example, the first fasteners 16 and the second fasteners 17 may form “snaps” as known to one having ordinary skill in the art. The first fasteners 16 may be connected to the first end 11 and/or the first side 21 of the garment 10, and/or the second fasteners 17 may be connected to the second end 12 and/or the second side 22 of the garment 10. The present invention is not limited to specific locations of the first fasteners 16 or the second fasteners 17.

As shown in FIG. 7, in an embodiment, the first fasteners 16 may be male fasteners, and the second fasteners 17 may be female fasteners. Alternatively, the first fasteners 16 may be female fasteners, and the second fasteners 17 may be male fasteners. The male fasteners may extend in a direction generally outward from the garment 10, and/or the female fasteners may extend in a direction generally inward into the garment 10. The male fasteners may insert into the female fasteners to connect the first end 11 of the garment 10 to the second end 12 of the garment 10, such as, for example, to wear the garment 10 on the neck of the user.

For example, if the first fasteners 16 are male fasteners and the second fasteners 17 are female fasteners, each of the first fasteners 16 may have a groove 46, and/or each of the second fasteners 17 may have a lip 47. The groove 46 may extend in a direction generally inward into the first fasteners 16, and/or the lip 47 may extend in a direction generally outward from the second fasteners 17. As another example, if the first fasteners 16 are female fasteners and the second fasteners 17 are male fasteners, each of the first fasteners 16 may have the lip 47, and/or each of the second fasteners 17 may have the groove 46. The groove 46 may extend in a direction generally inward into the second fasteners 16, and/or the lip 47 may extend in a direction generally outward from the first fasteners 17.

The lip 47 may insert into the groove 46 to connect the first fasteners 16 to the second fasteners 17. Then, the first fasteners 16 may be removed from the second fasteners 17 to separate the first end 11 of the garment 10 from the second end 12 of the garment 10, such as, for example, to remove the garment 10 from the neck of the user. For example, pulling the first end 11 of the garment 10 away from the second end 12 of the garment 10 may separate the lip 47 from the groove 46 to separate the first fasteners 16 from the second fasteners 17.

As shown in FIGS. 2, 5, 6 and 8, the garment 10 may have additional fasteners 18. As shown in FIGS. 2, 5, 6 and 8, in an embodiment, the additional fasteners 18 may be located at the second end 22 of the garment 10. The additional fasteners 18 may be located at a greater distance from the second edge 32 than the second fasteners 17. The additional fasteners 18 may be a same type of fastener as the second fasteners 17. For example, if the second fasteners 17 are female fasteners, the additional fasteners 18 may be female fasteners. If the second fasteners 17 are male fasteners, the additional fasteners 18 may be male fasteners.

In another embodiment, the additional fasteners 18 may be located at the first end 21 of the garment 10. The additional fasteners 18 may be located at a greater distance from the first edge 31 than the first fasteners 16. The additional fasteners 18 may be same type of fastener as the first fasteners 16. For example, if the first fasteners 16 are male fasteners, the additional fasteners 18 may be male fasteners. If the first fasteners 16 are female fasteners, the additional fasteners 18 may be female fasteners.

As shown in FIG. 6, if the first fasteners 16 are connected to the second fasteners 17, the garment 10 may have a first radius 50. As shown in FIG. 8, the additional fasteners 18 may enable the garment 10 to have a second radius 51 which may be a different distance than the first radius 50. For example, as previously set forth, if the additional fasteners 18 are located at the second end 22 of the garment 10, the additional fasteners 18 may be located at a greater distance from the second edge 32 than the second fasteners 17. As a result, if the first fasteners 16 connect to the additional fasteners 18 instead of the second fasteners 17, the garment 10 may have the second radius 51 instead of the first radius 50, and/or the second radius 51 may be a smaller distance than the first radius 50.

As another example, as previously set forth, if the additional fasteners 18 are located at the first end 21 of the garment 10, the additional fasteners 18 may be located at a greater distance from the first edge 31 than the first fasteners 16. As a result, if the second fasteners 17 connect to the additional fasteners 18 instead of the first fasteners 16, the garment 10 may have the second radius 51 instead of the first radius 50, and/or the second radius 51 may be a smaller distance than the first radius 50. The present invention is not limited to a specific number of additional fasteners 18. Any number of the additional fasteners 18 may be provided at various distances from the adjacent edge to enable the garment to have any number of various radii.

As shown in FIGS. 9-14, the first end 11 of the garment 10 may connect to the second end 12 of the garment using hook-and-loop fasteners, such as, for example, velcro (trademark of Velcro Industries B.V.). For example, the garment 10 may have a first hook-and-loop portion 56 and/or a second hook-and-loop portion 57. For example, the first hook-and-loop portion 56 may be a hook portion which may have hooks which may extend in a direction generally outward from the first end 11 of the garment 10. The second hook-and-loop portion 57 may be a loop portion which may have loops which may be smaller than the hooks and/or which may extend in a direction generally outward from the second end 12 of the garment 10. Alternatively, the first hook-and-loop portion 56 may be the loop portion and the second hook-and-loop portion 57 may be the hook portion. The first hook-and-loop portion 56 may be connected to the first end 11 and/or the first side 21 of the garment 10, and/or the second hook-and-loop portion 57 may be connected to the second end 12 and/or the second side 22 of the garment 10.

If the first hook-and-loop portion 56 contacts the second hook-and-loop portion 57, the hooks may insert into the loops to connect the first hook-and-loop portion 56 to the second hook-and-loop portion 57. If the user pulls the first hook-and-loop portion 56 away from the second hook-and-loop portion 57, the hooks may separate from the loops to separate the first hook-and-loop portion 56 from the second hook-and-loop portion 57.

The first hook-and-loop portion 56 may connect to the second hook-and-loop portion 57 to connect the first end 11 of the garment 10 to the second end 12 of the garment 10, such as, for example, to wear the garment 10 on the neck of the user. Subsequently, the first hook-and-loop portion 56 may be separated from the second hook-and-loop portion 57 to separate the first end 11 of the garment 10 from the second end 12 of the garment 10, such as, for example, to remove the garment 10 from the neck of the user. For example, the user pulling the first end 11 of the garment 10 away from the second end 12 of the garment 10 may separate the first hook-and-loop portion 56 from the second hook-and-loop portion 57.

The garment 10 having the first hook-and-loop portion 56 and the second hook-and-loop portion 57 may have a radius 60. The first hook-and-loop portion 56 and the second hook-and-loop portion 57 may enable the user to establish and/or adjust the radius 60. For example, the user may contact the first hook-and-loop portion 56 to a first area of the second hook-and-loop portion 57. The first area of the second hook-and-loop portion 57 may be adjacent to the second edge 32 of the garment 10. At another time, the user may contact the first hook-and-loop portion 56 to a second area of the second hook-and-loop portion 57. The second area of the second hook-and-loop portion 57 may be located at a greater distance from the second edge 32 than the first area of the second hook-and-loop portion 57. As a result, the radius 60 of the garment 10 may be a smaller distance if the first hook-and-loop portion 56 is connected to the second area of the second hook-and-loop portion 57 instead of the first area of the hook-and-loop portion 57. The present invention is not limited to a specific number of radii of the garment 10, and the first hook-and-loop portion 56 and the second hook-and-loop portion 57 may be used to establish any number of radii of the garment 10.

As generally illustrated in FIGS. 15 and 16, the garment 10 may have an insert 65 located within the garment 10. The insert 65 may be located between the first side 21 of the garment 10 and the second side of the garment 10. The insert may extend from a position adjacent to the first edge 31 to a position adjacent to the second edge 32. The insert 65 may extend from a position adjacent to the third edge 33 to a position adjacent to the fourth edge 34. In an embodiment, the insert 65 may have a rectangular shape. In an embodiment, the garment 10 may have three layers, namely the first side 21, the second side 22 and the insert 65. In an embodiment, the first side 21, the second side 22 and the insert 65 may be the only layers of the garment 10. The insert 65 may provide support for the garment 10. For example, the insert 65 may maintain contact of the first side 21 of the garment 10 with the neck of the user.

Thread 66 may connect the first side 21 of the garment 10 to the second side 22 of the garment 10. For example, stitching may connect the first side 21 of the garment 10 to the second side 22 of the garment 10 with repetitive loops of the thread 66 which extend through the first side 21 of the garment 10 and the second side 22 of the garment 10.

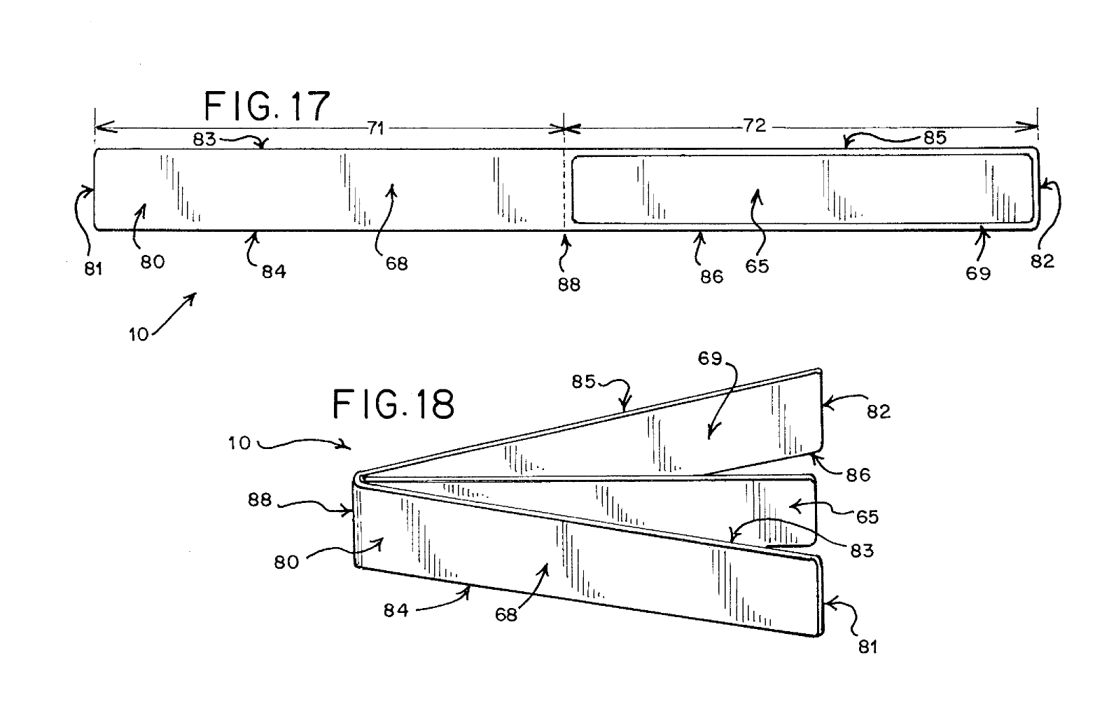

As shown in FIGS. 17 and 18, the first side 21 and the second side 22 of the garment 10 may be formed from one integral piece of material 80. For example, the first side 21 of the garment 10 may be integral with the second side 22 of the garment 10. In an embodiment, the one integral piece of material 80 may be rectangular. The one integral piece of material 80 may have any shape, and the present invention is not limited to a specific shape of the one integral piece of material 80.

The one integral piece of material 80 may have a first edge 81 and a second edge 82, and the first edge 81 of the one integral piece of material 80 may be located in a position opposite to the second edge 82 of the one integral piece of material 80. The first edge 81 of the one integral piece of material 80 may be parallel to the second edge 82 of the one integral piece of material 80. In an embodiment, the first edge 81 and the second edge 82 of the one integral piece of material 80 may be approximately the same length. The one integral piece of material 80 may have a midline 88 such that a distance 71 from the midline 88 to the first edge 81 may be approximately equal to a distance 72 from the midline 88 to the second edge 82.

The one integral piece of material 80 may have a first portion 68 and a second portion 69. The first portion 68 may extend from the midline 88 to the first edge 81 of the one integral piece of material 80. The second portion 69 may extend from the midline 88 to the second edge 82 of the one integral piece of material 80. The first portion 68 may be defined by the midline 88, the first edge 81, an upper edge 83 and a lower edge 84. The second portion 69 may be defined by the midline 88, the second edge 81, an upper edge 85 and a lower edge 86. The first portion 68 and the second portion 69 may have substantially the same dimensions, such as, for example, the same size and/or the same shape.

The upper edge 83 of the first portion 68 may be co-linear with the upper edge 85 of the second portion 69. The lower edge 84 of the first portion 68 may be co-linear with the lower edge 86 of the second portion 69. In an embodiment, the first portion 68 and the second portion 69 may have the same size and/or the same shape. In an embodiment, the first portion 68 and/or the second portion 69 may have a rectangular shape. For example, the upper edge 83 of the first portion 80 and the upper edge 85 of the second portion 69 may be perpendicular to the first edge 81 and/or the second edge 82 of the one integral piece of material 80. The lower edge 84 of the first portion 80 and/or the lower edge 86 of the second portion 69 may be perpendicular to the first edge 81 and/or the second edge 82 of the one integral piece of material 80. The upper edge 83 of the first portion 80 and the upper edge of the second portion 69 may be approximately the same length, and/or the lower edge 84 of the first portion 80 and/or the lower edge 86 of the second portion 69 may be approximately the same length.

As shown in FIGS. 17 and 18, the garment 10 may be formed by placing the insert 10 between the first portion 68 and the second portion 69 of the one integral piece of material 80. The one integral piece of material 80 may be folded at the midline 88 to enable connection of the first edge 81 of the one integral piece of material 80 to the second edge 82 of the one integral piece of material 80. The upper edge 83 of the first portion 68 may be connected to the upper edge 85 of the second portion 69, and/or the lower edge 84 of the first portion 68 may be connected to the lower edge 86 of the second portion 69. Connection of the first edge 81 to the second edge 82, connection of the upper edge 83 of the first portion 68 to the upper edge 85 of the second portion 69, and/or connection of the lower edge 84 of the first portion 68 to the lower edge 86 of the second portion 69 may enclose the insert 65 within the garment 10.

Connection of the first edge 81 to the second edge 82, connection of the upper edge 83 of the first portion 68 to the upper edge 85 of the second portion 69, and/or connection of the lower edge 84 of the first portion 68 to the lower edge 86 of the second portion 69 may be performed using any means known to one having ordinary skill in the art. In an embodiment, stitching may be used to connect the first edge 81 to the second edge 82, the upper edge 83 of the first portion 68 to the upper edge 85 of the second portion 69, and/or the lower edge 84 of the first portion 68 to the lower edge 86 of the second portion 69. For example, the thread 66 shown in FIGS. 15 and 16 may connect the first edge 81 to the second edge 82. The stitching may connect the first edge 81 to the second edge 82 with repetitive loops of the thread 66 which extend through the first edge 81 to the second edge 82. As a further example, the thread 66 shown in FIGS. 15 and 16 may connect the upper edge 83 of the first portion 68 to the upper edge 85 of the second portion 69. The stitching may connect the upper edge 83 of the first portion 68 to the upper edge 85 of the second portion 69 with repetitive loops of the thread 66 which extend through the upper edge 83 of the first portion and the upper edge 85 of the second portion 69. As yet another example, the thread 66 shown in FIGS. 15 and 16 may connect the lower edge 84 of the first portion 68 to the lower edge 86 of the second portion 69. The stitching may connect the lower edge 84 of the first portion 68 to the lower edge 86 of the second portion 69 with repetitive loops of the thread 66 which extend through the lower edge 84 of the first portion 68 and the lower edge 86 of the second portion 69.

In another embodiment, adhesive may be used to connect the first edge 81 to the second edge 82, the upper edge 83 of the first portion 68 to the upper edge 85 of the second portion 69, and/or the lower edge 84 of the first portion 68 to the lower edge 86 of the second portion 69. The present invention is not limited to a specific means of connecting the first edge 81 to the second edge 82, the upper edge 83 of the first portion 68 to the upper edge 85 of the second portion 69, and/or the lower edge 84 of the first portion 68 to the lower edge 86 of the second portion 69.

Referring again to FIGS. 17 and 18, the first edge 81 of the one integral piece of material 80 may connect to the second edge 82 of the one integral piece of material 80 to form the first end 21 of the garment 10 or the second end 22 of the garment 10. If the first edge 81 and the second edge 82 of the one integral piece of material 80 form the first end 21 of the garment 10, the midline 88 of the one integral piece of material 80 may form the second end 22 of the garment 10. If the first edge 81 and the second edge 82 of the one integral piece of material 80 form the second end 22 of the garment 10, the midline 88 of the one integral piece of material 80 may form the first end 21 of the garment 10.

The upper edge 83 of the first portion 68 may connect to the upper edge 83 of the second portion 69 to form the third edge 33 of the garment 10 or the fourth edge 34 of the garment 10. If the upper edge 83 of the first portion 68 and the upper edge 83 of the second portion 69 form the third edge 33 of the garment 10, the lower edge 84 of the first portion 68 and the lower edge 86 of the second portion 69 may form the fourth edge 34 of the garment 10. If the upper edge 83 of the first portion 68 and the upper edge 83 of the second portion 69 form the fourth edge 34 of the garment 10, the lower edge 84 of the first portion 68 and the lower edge 86 of the second portion 69 may form the third edge 33 of the garment 10.

The one integral piece of material 80 and the insert 65 may be made from any material. The one integral piece of material 80 and the insert 65 may be made from a material which may enable the garment 10 to be washed using a standard washing machine and/or may be hand-washed. For example, the garment 10 may be washed using a standard washing machine on a delicate setting. In an embodiment, the one integral piece of material 80 and/or the insert 65 may be a woven material. As a result, the first side 21 of the garment 10, the second side of the garment 10, and/or the insert 65 may be a woven material. In a preferred embodiment, both the one integral piece of material 80 and the insert 65 may be 100% cotton. As a result, the first side 21 of the garment 10, the second side 22 of the garment 10 and the insert 65 may be 100% cotton. In an embodiment, the one integral piece of material 80 and/or the insert 65 may be a man-made material.

Buttons, magnets, hooks, a buckle, laces and/or the like may be used instead of or to supplement the first fasteners 16, the second fasteners 17, the additional fasteners 18, the first hook-and-loop portion 56 and/or the second hook-and-loop portion 57. For example, buttons, magnets, hooks, a buckle, laces and/or the like may connect the first end 21 of the garment 10 to the second end 22 of the garment 10. The present invention is not limited to a specific embodiment of connecting means which connect the first end 21 of the garment 10 to the second end 22 of the garment 10.

The user may determine that use of the garment 10 is desired, such as, for example, if the user is coughing, and may place the garment 10 around the neck of the user. Then, the first end 11 of the garment 10 may be connected to the second end 12 of the garment 10 to maintain a position of the garment 10 on the neck of the user. The garment 10 may provide warmth to the neck of the user. For example, maintaining contact of the garment 10 with the neck of the user may provide warmth to the neck of the user. The three layers of the garment 10, namely the first side 21 of the garment 10, the insert 65, and the second side 22 of the garment may provide the warmth. The warmth may thin the mucus in the throat, and thinning the mucus in the throat may suppress and/or may prevent coughing and/or may naturally lubricate the throat. The user may subsequently determine that use of the garment 10 is no longer desired, such as, for example, if coughing ceases, and may separate the first end 11 of the garment 10 from the second end 12 of the garment 10. Then, the user may remove the garment 10 from the neck of the user.

It should be understood that various changes and modifications to the presently preferred embodiments described herein will be apparent to those skilled in the art. Such changes and modifications may be made without departing from the spirit and scope of the present invention and without diminishing its attendant advantages. It is, therefore, intended that such changes and modifications be covered by the appended claims.

1. A method for suppressing coughing as a result of mucus in a throat within a neck of a user, the method comprising the steps of:
placing a garment having a substantially rectangular shape to align the garment around the neck at the throat of the user to warm the neck and thin the mucus in the throat wherein the garment has a first side, a second side and an insert wherein the first side is located in a position opposite to the second side wherein the insert is substantially rectangular and located within the garment between the first side and the second side wherein the neck of the user contacts the first side and further wherein the first side has a first hook-and-loop portion and the second side has a second hook-and-loop portion; and
connecting the first hook-and-loop portion to the second hook-and-loop portion to attach the first side to the second side and maintain the garment around the neck at the throat of the user.

2. (canceled)

3. (canceled)

4. (canceled)

5. (canceled)

6. (canceled)

7. The method of claim 1 further comprising the step of:
separating the first hook-and-loop portion from the second hook-and-loop portion to detach the first side from the second side.

8. The method of claim 1 further comprising the step of:
removing the garment from the neck of the user.

9. The method of claim 1 further comprising the step of:
adjusting a radius of the garment.

10. The method of claim 1 further comprising the step of:
providing thread to connect the first side to the second side.
